(19) United States
(12) Patent Application Publication
(10) Pub. No.: US 2010/0108950 A1
(43) Pub. Date: May 6, 2010

(54) METHOD FOR PREPARING AN AQUEOUS SUSPENSION OF CARBON NANOTUBES AND SUSPENSION THUS OBTAINED

(75) Inventor: Dominique Plee, Lons (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: Arkema France, Colombes (FR)

(21) Appl. No.: 12/596,533

(22) PCT Filed: Apr. 18, 2008
(86) PCT No.: PCT/FR08/50697

§ 371 Date: Oct. 19, 2009

(30) Foreign Application Priority Data
Apr. 20, 2007 (FR) 07 02874

(51) Int. Cl. H01B 1/24; A61K 8/72; C08K 3/04

(52) U.S. Cl. 252/511; 523/105; 524/496; 524/612; 977/750; 977/904; 977/932

(57) ABSTRACT

The invention relates to a method for preparing an aqueous suspension of carbon nanotubes, that comprises: contacting in an aqueous medium said nanotubes with at least one dispersant comprising a copolymer including at least one anionic hydrophile monomer and at least one monomer containing at least one aromatic group substituted by at least one chain containing one or more oxygen atoms, said chain linking the aromatic group to the unsaturated or cyclic chain of the monomer capable of opening during the formation of the copolymer, the weight ratio between the dispersant and the carbon nanotubes ranging from 0.6:1 to 1.9:1; and mechanically processing the mixture thus obtained using ultrasounds or a rotor-stator system or by passing it in a bead or ball grinder, the invention also relates to the suspension thus obtained and to the uses thereof.

The present invention relates to an aqueous suspension of carbon nanotubes, its method of preparation and its uses.

Carbon nanotubes (or CNT) are known and have particular crystal structures, a tubular shape, hollow and closed, comprising atoms arranged uniformly in pentagons, hexagons and/or heptagons, obtained from carbon. CNT generally consists of one or more wound graphene sheets. A distinction is thus drawn between Single Wall Nanotubes or SWNT and Multi Wall Nanotubes or MWNT.

CNT is available on the market or can be prepared by known methods. Several methods are available for synthesizing CNT, in particular electrical discharge, laser ablation and chemical vapor deposition or CVD, which is suitable for manufacturing large quantities of carbon nanotubes and therefore obtaining them at a production cost compatible with their massive use. This process consists in particular in injecting a carbon source at relatively high temperature on a catalyst which may itself consist of a metal such as iron, cobalt, nickel or molybdenum, supported by an inorganic solid such as alumina, silica or magnesia. The carbon sources may comprise methane, ethane, ethylene, acetylene, ethanol, methanol, or even a mixture of carbon monoxide and hydrogen (HIPCO process).

Thus, application WO 86/03455A1 by Hyperion Catalysis International Inc. describes in particular the synthesis of CNT. More particularly, the method comprises the contacting of a particle based on metal such as in particular, iron, cobalt or nickel, with a gaseous compound based on carbon, at a temperature of between about 850° C. and 1200° C., the proportion by dry weight of the carbon-based compound with regard to the metal based particle being at least about 100:1.

From a mechanical standpoint, CNTs have both excellent rigidity (measured by the Young's modulus), comparable to that of steel, while being extremely lightweight. Moreover, they have excellent properties of electrical and thermal conductivity, making them suitable for use as additives to confer these properties on various materials, in particular macromolecular, such as polyamides, polycarbonate, polyesters, polystyrene, polyethelether ketones and polyethylene imine.

However, CNTs prove to be difficult to handle and to disperse, due to their small size, their powdery property and possibly, when they are obtained by the CVD technique, their tangled structure, which is more pronounced when their mass productivity is increased for the purpose of improving production and reducing the residual ash content. The presence of strong Van der Waals interactions between single wall nanotubes is also detrimental to their dispersibility and the stability of the suspensions obtained.

To remedy the poor dispersibility of CNTs, which substantially affects the characteristics of the composites that they form with the polymer matrices into which they are introduced, various solutions have already been proposed in the prior art. Among them mention can be made of sonication, but which only has a temporary effect, or ultrasonication, which has the effect of partly cutting the nanotubes and creating oxygenated functions which may alter some of their properties.

It has also been suggested to prepare mixtures in a solvent of CNT with dispersants such as surfactants including sodium dodecylsulfate (EP-1 495 171; VIGOLO B. et al, Science, 290 (2000), 1331; WANG J. et al, J. of Chem. Society, 125 (2003), 2408; MOORE, V. C. et al, Nanoletters, 3, (2003), 2408). However, the latter are unsuitable for dispersing large quantities of CNT, and satisfactory dispersions can only be obtained for CNT concentrations of less than 2 or 3 g/l. Furthermore, the surfactants are liable to desorb completely from the CNT surface during the dialysis step generally employed to remove the excess surfactant in the solution, with the effect of destabilizing the suspension obtained.

Similarly, it has been suggested to use water soluble polymers such as gum arabic or a copolymer of styrene and t-butyl acrylate to disperse single wall carbon nanotubes in water or ethanol, respectively (WO 02/76888 and WO 2005/073305). However, the first of these methods requires the use of large quantities of polymer and is unsuitable for stabilizing more than 0.5 g/l of CNT.

In other documents, (WO 02/016257 and WO 2004/097853), it has been suggested to disperse CNT using copolymers of maleic acid and styrene sulfonic acid. Here also, obtaining a concentrated dispersion of CNT requires the use of large quantities of dispersant.

Another solution, proposed in particular in applications EP-1 359 121 and EP-1 359 169, consisted in preparing a dispersion of CNT in a solvent and a monomer and carrying out an in situ polymerization to yield functionalized CNT.

Similarly, application FR-2 870 251 discloses a composite material obtained by introducing, into a polymer matrix, a pre-composite itself comprising carbon nanotubes (CNT) treated by a compatibilizer which is a polymer containing at least one B1 block comprising an acid monomer and/or anhydride, such as an acrylic monomer, and a styrene, (meth)acrylate or (meth)acrylamide monomer, and optionally a B2 block compatible with the polymer matrix, such as a vinyl, vinyldiene, diene or olefinic block and in particular a styrene based block. The B1 block is preferably polymerized in the presence of CNT dispersed in a solvent which may be water or an organic solvent, with mechanical stirring or sonication. The product obtained can be used in particular as an additive in latex. Example 4 O in this document also describes the formation of an acrylic/styrene copolymer in the presence of CNT in dioxane. The pre-composite obtained is well dispersed in toluene and can then be incorporated in polystyrene.

These solutions are nevertheless complex and may prove to be costly depending on the products used. Moreover, the grafting operations are liable to damage the structure of the nanotubes, and consequently, their electrical and/or mechanical properties (GARG A. et al, Chem. Phys. Lett. 295, (1998), 273).

Applications WO 03/050332 and WO 03/106600 describe dispersions of carbon nanotubes, particularly in water, in the presence of copolymers which may contain alkyl methacrylate motifs. These dispersions may, for example, be prepared using ultrasound or a colloid mill. This solution generally yields a rather mediocre dispersion, probably due to the poor affinity of the polymer for the nanotubes.

Furthermore, application WO 2005/07335 Ben Gourion suggests the use of a block copolymer to disperse carbon nanotubes in a selective solvent of one of the blocks, such as ethanol or isopropanol in the case of a styrene and t-butyl acrylate copolymer, advantageously using ultrasound.

The Applicant has now discovered that the choice of a particular copolymer mixed with carbon nanotubes in the presence of ultrasound or using a rotor-stator system offered a simple, inexpensive means, using little solvent, for uniformly dispersing these carbon nanotubes in water up to nanotube concentrations of 10 g/l, without substantially affecting their mechanical and electrical properties.

Such a copolymer has been described in particular in U.S. Pat. No. 6,093,764 as a dispersant of a mineral filler, such as calcium carbonate, in aqueous medium. To the knowledge of the Applicant, it has never yet been suggested to use this copolymer to disperse carbon nanotubes in water in the presence of ultrasound or using a shear system.

The object of the present invention is therefore a method for preparing an aqueous suspension of carbon nanotubes, comprising:
contacting said nanotubes in aqueous medium with at least one dispersant consisting of a copolymer containing at least one anionic hydrophilic monomer and at least one monomer containing at least one aromatic group substituted by at least one chain containing one or more oxygen atoms, said chain linking the aromatic group to the unsaturated or cyclic chain of the monomer capable of opening during the formation of the copolymer, the weight ratio of the dispersant to the carbon nanotubes employed ranging from 0.6:1 to 1.9:1 and
mechanically processing the mixture thereby obtained with ultrasound or using a rotor-stator system or by passage through a ball mill.

The inventive method serves to obtain suspensions which are stable for several days at ambient temperature and which, in particular do not contain grains of over 30 μm or even or over 20 μm and/or containing a concentration of carbon nanotubes in the supernatant of the suspension at least equal to 80% of the concentration used, and for carbon nanotube concentrations ranging from 1 to 50 g/l, in particular from 5 to 20 g/l and more particularly from 10 to 20 g/l.

In the context of the present invention, “aqueous medium” means any medium containing at least water as continuous phase, optionally mixed with at least one water miscible solvent such as alcohols, including ethanol and/or ketones including acetone and methylethylketone and/or in which at least one oil can be dispersed. The expression “aqueous medium” thus also covers latex and oil-in-water emulsions, for example. It is preferable for this medium to consist of the abovementioned components and advantageously for it to contain water only.

The carbon nanotubes (here below CNT) usable according to the invention may be of the single wall, double wall or multiwall type. Double wall CNT can in particular be prepared as described by Flahaut et al in Chem. Com. (2003), 1442. Multi wall CNT can be prepared as described in document WO 03/02456.

The CNT employed according to the invention normally have a diameter of between 0.1 and 100 nm, preferably between 0.4 and 50 nm and even better between 1 and 30 nm and advantageously a length of 0.1 to 10 μm. Their length/diameter ratio is advantageously higher than 10 and usually higher than 100. Their specific surface area is, for example, between 100 and 300 m²/g and their apparent density may be between 0.05 and 0.5 g/cm³ and more particularly between 0.1 and 0.2 g/cm³. Multiwall carbon nanotubes may, for example, comprise 5 to 15 flakes and more preferably 7 to 10 flakes.

One example of untreated nanotubes is available in particular on the market from Arkema under the trade name Graphistrength® C100.

These nanotubes can be purified and/or oxidized and/or ground, before they are used in the inventive method.

CNT can in particular be ground hot or cold by known techniques applied in apparatus such as ball mills, hammer mills, pan mills, cutting mills, gas jet or any other grinding system capable of reducing the size of the tangled network of CNT. It is preferable for this grinding step to be carried out using a gas jet grinding technique, in particular in an air-jet pulverizer.

CNT can be purified by washing with a sulfuric acid solution, in order to strip them of any residual mineral and metallic impurities, resulting from their preparation method. The weight ratio of CNT to sulfuric acid may in particular be between 1:2 and 1:3, bounds included. The purification operation can also be carried out at a temperature between 90 and 120° C., for example for a period of 5 to 10 hours. This operation can advantageously be followed by steps of rinsing with water and drying of the purified CNT.

CNT are advantageously oxidized by contacting them with a sodium hypochlorite solution containing 0.5 to 15% by weight of NaOCl and preferably 1 to 10% by weight of NaOCl, for example in a weight ratio of CNT to sodium hypochlorite of between 1:0.1 and 1:1. The oxidation is advantageously carried out at a temperature lower than 60° C. and preferably at ambient temperature, for a period ranging from a few minutes to 24 hours. This oxidation operation can advantageously be followed by steps of filtration and/or centrifugation, washing and drying of the oxidized CNT.

In a first step of the inventive method, the CNT (untreated or ground and/or purified and/or oxidized) are contacted with a dispersant consisting of a copolymer containing at least one anionic hydrophilic monomer and at least one monomer containing at least one aromatic group substituted by at least one chain containing one or more oxygen atoms, said chain linking the aromatic group to the unsaturated or cyclic chain of the monomer capable of opening during the formation of the copolymer. Preferably, this copolymer contains no other monomer than the abovementioned two.

It is preferable according to the invention for the anionic hydrophilic monomer to be selected from ethylenically unsaturated monomers having at least one carboxylic acid function, such as acrylic, diacrylic, methacrylic, crotonic, isocrotonic, cinnamic, maleic, fumaric, dimethylfumaric, itaconic, citraconic, vinylbenzoic, acrylamidoglycolic acids, carboxylic anhydrides having one vinyl bond such as maleic anhydride, and their salts and mixtures thereof.

In a less preferred alternative, the anionic hydrophilic monomer may be selected from ethylenically unsaturated monomers having at least one sulfonic acid function, such as acrylamidopropanesulfonic acid, 2-acrylamido 2-methylpropanesulfonic acid, styrene sulfonic acid, vinylsulfonic acid, vinylbenzene sulfonic acid, their salts and mixtures thereof.

The salts of the abovementioned monomers may in particular be alkali metal salts, such as sodium or potassium salts; salts of alkaline earth metals, in particular of magnesium and calcium; ammonium salts; salts of primary, secondary or tertiary amines, for example stearylamine, ethanolamine, mono- and diethylamine; or aluminum salts.

The copolymer preferably comprises 10 to 99% by weight and more preferably 50 to 97% by weight of anionic hydrophilic monomer and 1 to 90% by weight, preferably 3 to 50% by weight of monomer containing an aromatic group.

In the monomer containing at least one aromatic group, the chain containing one or more oxygen atoms may in particular constitute a poly(alkylene glycol) group, which may itself be a poly(propylene glycol) or poly(ethylene glycol) group, or a mixture of these two groups. A preferred example of such a monomer is arylether (meth)acrylate of poly(alkylene glycol). It is also preferable for the aryl group of the arylether to be a phenyl group. This aryl group may further be substituted by at least one alkyl and/or arylalkyl radical such as a tristyryl radical. The poly(alkylene glycol) is in this case preferably a poly(ethylene glycol) group. The number of oxyalkylene motifs may range from 5 to 100 and preferably from 10 to 50.

A preferred dispersant for use in the present invention is ethoxylated phenol tristyryl methacrylate containing 25 moles of ethylene oxide, which is available in particular from Coatex in the form of an aqueous solution containing 25% by weight of polymer. Such a polymer is described in particular in U.S. Pat. No. 6,093,764. Another dispersant suitable for use in the present invention is sold by Rohm & Haas under the trade name OROTAN® 731K.

According to a preferred embodiment of the invention, the weight ratio of dispersant to the carbon nanotubes employed is between 0.6:1 and 1:1. It is also preferable for the total mass of dispersant and carbon nanotubes to account for 0.1 to 5% and more preferably for 0.5 to 2% by weight of aqueous medium.

In the second step of the inventive method, the mixture of CNT and the dispersant is subjected to a mechanical processing selected from ultrasound, processing using a rotor-stator system or passage through a ball mill.

In the case in which ultrasonic treatment is carried out, it is preferable for this to be carried out for more than 10 minutes at a frequency of at least 20 kHz, for example for 20 to 40 minutes at this frequency. The Applicant has demonstrated that the passage of the carbon nanotube suspension under ultrasound constitutes a preferred alternative treatment in the case in which a film is to be formed subsequently from this suspension.

Furthermore, an example of a rotor-stator system suitable for use in the present invention generally comprises a rotor actuated by a motor and provided with fluid guide systems perpendicular to the rotor shaft, such as blades or vanes arranged substantially radially or a flat disk provided with peripheral teeth, said rotor optionally being provided with a toothed wheel, and a stator arranged concentrically about the rotor, and at a short distance outside same, said stator being equipped on at least a portion of its circumference with openings, made for example in a grid or mutually defining one or more rows of teeth, which are adapted to the passage of the fluid drawn through the rotor and ejected by the guide systems toward said openings. One or more of the abovementioned teeth may be provided with sharp edges. The fluid is thus subjected to a high shear, both in the air gap between the rotor and the stator and through the openings made in the stator.

Such a rotor-stator system is sold in particular by Silverson under the trade name Silverson® L4RT.

Another type of rotor-stator system is sold by Ika-Werke under the trade name Ultra-Turrax®.

Other rotor-stator systems consist of colloid mills, deflocculating turbines and high shear mixers of the rotor-stator type, such as the apparatus sold by Ika-Werke or by Admix.

According to the invention, it is preferable for the speed of the rotor to be set to at least 1000 rpm and preferably to at least 3000 rpm or even to at least 5000 rpm. Furthermore, it is preferred that the width of the air gap between the rotor and the stator be less than 1 mm and preferably less than 200 mm, more preferably less than 100 μm and even better, less than 50 μm or even less than 40 μm. Furthermore, the rotor-stator system used according to the invention advantageously confers a shear of 1000 to 10⁹ s⁻¹.

According to a preferred embodiment, the inventive method is implemented in such a way that the concentration of carbon nanotubes before passage through the rotor-stator is at least 15 g/l, or even at least 20 g/l, and the nanotubes are then diluted with water after passage through the rotor-stator. It has in fact been observed that by working on more viscous suspensions the power consumption in the apparatus was higher and the nanotube suspension obtained after shear was more stable.

The present invention also relates to the suspension which can be obtained by the method as described above.

The suspension according to the invention can be used in particular for reinforcing polymer matrices; for fabricating packing material for electronic components (intended for example for electromagnetic shielding and/or antistatic dissipation), such as cases of mobile telephones, computers, electronic apparatus on board motor, rail or air vehicles; for fabricating inks for electrical connection between two electronic components; or for fabricating medical instruments, fuel hoses (gasoline or diesel), adhesives, antistatic coatings, thermistors, or electrodes of light emitting diodes, photovoltaic cells or supercapacitances.

The present invention therefore also relates to the use of the suspension as defined previously for the abovementioned purposes.

The invention is now illustrated by the following nonlimiting examples.

### EXAMPLES

### Example 1

### Preparation of a Sample of Untreated CNT

A sample of CNT is prepared by chemical vapor deposition (CVD) from ethylene at 650° C., which is passed on a catalyst consisting of iron supported on alumina. The reaction product contains an ash content, measured by ignition loss at 650° C. in air, of 7%. This sample, which is denoted below as CNT1, contains 3% of Fe₂O₃ and 4% of Al₂O₃, determined by chemical analysis.

### Example 2

### Preparation of a Sample of Purified CNT

18.5 g of CNT1, obtained as described in Example 1, is subjected to a purification operation in 300 ml of 14% sulfuric acid for 8 hours at 103° C. Once washed with water and dried a product is obtained, identified as CNT2, containing an ash content of 2.6% (including 2.5% of Fe₂O₃ and 0.1% Al₂O₃, determined by chemical analysis).

### Example 3

### Preparation of a Sample of Oxidized CNT

Two solutions of 100 ml of sodium hypochlorite containing 2% and 5% by weight are prepared, respectively, to which 5 g of CNT1 prepared as described in Example 1 are added. After 4 h with magnetic stirring at ambient temperature, the samples are filtered, washed and dried. They are respectively denoted below as CNT3 and CNT4. The measurement of the surface functions by ESCA shows that, while the aluminum content is not decreased, the proportion of oxygenated functions is much higher than in sample CNT1.

### Example 4

### Preparation of Aqueous Suspensions of Untreated CNT in the Presence of Dispersant

Various suspensions of CNT were prepared by the following method: 4 g of a solution of oxyethylated tristyrylphenol methacrylate (25 OE) containing 25% of active matter, supplied by Coatex, was added to a 125 ml beaker, and the volume made up to 100 ml with softened water. 1 g of CNT was added, and the mixture then subjected to ultrasound at a frequency of 20 kHz, using a Vibracell apparatus from Bioblock, having a declared electrical capacity of 300 W.

After 5 days of rest at ambient temperature, the concentration of nanotubes in the supernatant was measured and the state of the dispersion observed, in particular the possible presence of grains.

The various suspensions tested, and the results obtained, are given in Table 1 below.

TABLE 1
Evaluation of aqueous suspension of CNT1/dispersant

| | | | CNT | |
|---|---|---|---|---|
| | | | concentration | |
| | Type of | Sonication | in supernatant | Visual |
| Example | CNT | type (min) | (g/l) | appearance |
| 4A | CNT1 | 10 | 5.7 | Few grains |
| 4B | CNT1 | 30 | 8.3 | Few grains |
| 4C | CNT2 | 30 | 9.5 | Few grains |
| 4D | CNT3 | 30 | 9.9 | Very few |
| | | | | grains |

As this table shows, the suspensions according to the invention, having a weight ratio of CNT to dispersant of 1:1, yield suspensions that have few or no grains and a good CNT concentration in the supernatant, which is improved with longer ultrasonication time and/or higher density of oxygenated functions (created by the sodium hypochlorite), reflecting the good dispersion of the CNT in the water.

### Example 5 (Comparative)

### Suspensions with Different CNT/Dispersant Ratios

In a similar manner to Example 4, various suspensions of CNT (1 g) were prepared in water. In the presence of the same dispersant, and then subjected to 30 minutes of stirring with ultrasound.

The suspensions prepared, and the results obtained, are shown in Table 2 below.

TABLE 2
Evaluation of aqueous suspension of CNT/dispersant

| | | | CNT | |
|---|---|---|---|---|
| | | | concentration | |
| | Type of | Sonication | in supernatant | Visual |
| Example | CNT | type (min) | (g/l) | appearance |
| 5A | CNT1 | 2 | 6 | Presence of |
| | | | | grains |
| 5B | CNT1 | 2 | 7.2 | Presence of |
| | | | | grains |
| 5C | CNT2 | 0.5 | 3.5 | Presence of |
| | | | | grains |

It is thus found that a ratio of dispersant to CNT lower than or equal to 0.5:1 or higher than or equal to 2:1 does not serve to obtain a satisfactory dispersion. It is believed, without being necessarily bound by this theory, that the increase in this ratio destabilizes the suspension, probably by the formation of bridges between the particles, while its decrease has the same effect due to the lack of species capable of stabilizing the particles.

### Example 6

### Preparation Using the Rotor-Stator Mixer of an Aqueous Suspension of Purified CNT in the Presence of Dispersant

An aqueous suspension of CNT (4 g) of the CNT2 type, obtained as described in Example 2, was prepared in the presence of the same dispersant (4 g of active matter). This dispersion was mixed in a Silverson® L4RT rotor-stator system.

This apparatus comprises a hollow vertical rotor 31 mm in diameter and a concentric grid acting as a stator in 32 mm in diameter, the dispersion flowing radially from the inside to the outside of the apparatus. The speed of rotation is 7000 rpm, representing a peripheral speed of about 12 m/s.

The operation begins with a grid perforated with small square 5 mm sided holes to allow rapid pumping of the suspension, and continuous, after thickening of the suspension, with a grid perforated with small square 2 mm sided holes for 10 minutes. A dilution with water is then performed to obtain 10 g/l of carbon nanotubes. After 5 days of rest at ambient temperature, no grains are observed and the nanotube concentration in the supernatant is 9.8 g/l, or very close to the expected value.

This substantially produces the same result as with the sample 4D of Example 4, without the necessity of subjecting the CNT to oxidation with sodium hypochlorite.

### Example 7

### Preparation with the Rotor-Stator Mixer of an Aqueous Suspension of Oxidized CNT in the Presence of Dispersant

A solution of 100 ml of sodium hypochlorite is prepared containing 2.4% by weight, to which 5 g of CNT2 prepared as described in Example 2 is added. After 4 h with magnetic stirring at ambient temperature, the sample is filtered, washed and dried. It is subsequently denoted by CNT5.

An aqueous suspension of CNT is then prepared as described in Example 6, except that it is prepared with 50 g/l of CNT5 types of CNT and 50 g/l of dispersant, the suspension being diluted after passing through the Silverson® to obtain a CNT concentration of 10 g/l.

The film obtained by simple drying of this suspension is then observed and its conductivity measured using the 4-wire method. This method consists in measuring the conductivity using a system consisting of four parallel and horizontal copper wires, that is two outer wires connected to one of the poles and two inner wires connected to the other pole, the product to be tested being held by pressure on the wires.

The results obtained are given in Table 3 below.

TABLE 3
Properties of CNT5/dispersant film

| | Film | Film specific | Conductivity |
|---|---|---|---|
| Example | thickness (μm) | gravity | (S · cm⁻²) |
| 7A | 128 | 1.02 | 0.0026 |

It is thus observed that the suspension obtained according to the invention serves to obtain a fairly dense and conductive film despite the presence of dispersant, which could have been expected to hinder the flow of current. The current-potential curve plotted also appears to be quite linear.

### Example 8

### Comparison of Suspensions and Films Obtained from Various Polymers

Various suspensions were prepared by mixing 1 g of subsequently oxidized CNT2 with sodium hypochlorite in the conditions of Example 7 with various concentrations of an acrylic acid copolymer. The copolymers used were respectively a polymeric dispersant according to the invention (OROTAN® 731 K from Rohm & Haas) and a copolymer of acrylic acid and ethyl acrylate, having a molecular weight of 5000 (ACUMER® 2200 neutralized to pH 7, from Rohm & Haas) not according to the invention. These suspensions were then subjected to ultrasound on the same instrument as in Example 4, at the rate of 10 times 4 minutes with 15 minutes between each treatment, to allow the suspension to cool.

A few drops of the suspension are then taken and deposited in a layer on a sheet of PET which is then allowed to dry. The density and thickness of the film obtained are measured, and also the conductivity using the 4-wire method.

The suspensions prepared, and the results obtained, are shown in Table 4 below.

TABLE 4
Evaluation of CNT/polymer films on PET

| | | Polymer/ | Film | Film | |
|---|---|---|---|---|---|
| | Polymer | CNT | thickness | specific | Conductivity |
| Example | tested | ratio | (μm) | gravity | (S · cm⁻²) |
| 8A | ACUMER | 0.25 | 60 | 0.53 | 0.9 |
| | 2200 | | | | |
| 8B | OROTAN | 0.25 | 59 | 0.73 | 1 |
| | 731 K | | | | |
| 8C | OROTAN | 0.5 | 56 | 1.09 | 3 |
| | 731 K | | | | |
| 8D | ACUMER | 1 | 29 | 1.01 | 2 |
| | 2200 | | | | |

It is visually observed that OROTAN® 731 K, which is a dispersant in the context of the present invention, serves to form very smooth and self-supporting films on PET, which is not the case of the comparative polymer, while having good electrical conductivity properties, as shown in the table above.

The appearance of the suspensions is also evaluated visually (viscosity, presence or absence of grains using the North-Grindometer gauge from Gardner) and the CNT concentration in the supernatant after five days of rest at ambient temperature, and also the capacity of these suspensions to form a film on glass.

The results obtained are given in Table 5 below.

TABLE 5
Evaluation of suspension and films of CNT/polymer on glass

| | CNT | | |
|---|---|---|---|
| | concentration | | Appearance |
| | in supernatant | Appearance of | of film in |
| Example | (g/l) | suspension | glass |
| 8A | 10 | Viscous, many | No film |
| | | grains | |
| 8B | 8.9 | Fluid, very | Good film |
| | | few grains | |
| 8C | 9.2 | Fluid, very | Good film |
| | | few grains | |
| 8D | 10 | Fluid, very | No film |
| | | few grains | |
| 8E (ratio | 9.7 | Fluid, very | No film |
| OROTAN ®/CNT = 1) | | few grains | |
| 8F (ratio | 10 | Viscous, many | No film |
| OROTAN ®/CNT = 2) | | grains | |
| 8G (ratio | 10 | Viscous, very | No film |
| OROTAN ®/CNT = 3) | | few grains | |

It appears from this table that the polymeric dispersant according to the invention serves to obtain a CNT concentration in the supernatant close to the expected value (10 g/l) and fluid suspensions having few grains, reflecting the good dispersion of the CNT in the water. Furthermore, for polymer/CNT ratios of 0.25 to less than 1, it leads to the formation of good films on glass. On the contrary, the comparative polymer, which does not contain any aromatic monomer, does not improve the dispersion of the CNT in water and does not allow the formation of a film on glass.

1. A method for preparing an aqueous suspension of carbon nanotubes, comprising:
contacting said nanotubes in aqueous medium with at least one dispersant consisting of a copolymer containing at least one anionic hydrophilic monomer and at least one monomer containing at least one aromatic group substituted by at least one chain containing one or more oxygen atoms, said chain linking the aromatic group to the unsaturated or cyclic chain of the monomer capable of opening during the formation of the copolymer, the weight ratio of the dispersant to the carbon nanotubes employed ranging from 0.6:1 to 1.9:1 and
mechanically processing the mixture thereby obtained with ultrasound or using a rotor-stator system or by passage through a ball mill.

2. The method as claimed in claim 1, wherein the carbon nanotubes are capable of being obtained by a chemical vapor deposition process.

3. The method as claimed in either of claim 1, wherein the carbon nanotubes have a diameter ranging from 0.1 to 100 nm, preferably from 0.4 to 50 nm and, even better, from 1 to 30 nm.

4. The method as claimed in claim 1, wherein the carbon nanotubes have a length of 0.1 to 10 μm.

5. The method as claimed in claim 1, wherein the carbon nanotubes are untreated nanotubes, purified using a sulfuric acid solution, oxidized using a sodium hypochlorite solution and/or ground using an air-jet pulverizer.

6. The method as claimed in claim 1, wherein the anionic hydrophilic monomer is selected from ethylenically unsaturated monomers having at least one carboxylic acid function, carboxylic anhydrides having one vinyl bond, and their salts and mixtures thereof.

7. The method as claimed in claim 6, wherein the anionic hydrophilic monomer is selected from acrylic, diacrylic, methacrylic, crotonic, isocrotonic, cinnamic, maleic, fumaric, dimethylfumaric, itaconic. citraconic, vinvlbenzoic, acrylamidoglycolic acids and their mixtures.

8. The method as claimed in claim 6, wherein the anionic hydrophilic monomer is maleic anhydride.

9. The method as claimed in claim 1, wherein the chain containing one or more oxygen atoms is a poly(alkylene glycol) chain.

10. The method as claimed in claim 1, wherein the monomer containing at least one aromatic group consists of an arylether(meth)acrylate of (poly)alkylene glycol.

11. The method as claimed in claim 10, wherein the aryl group is a phenyl group.

12. The method as claimed in claim 10, wherein the aryl group is substituted by at least one alkyl and/or arylalkyl radical.

13. The method as claimed in claim 10, wherein the aryl group is substituted by at least one tristyryl radical.

14. The method as claimed in claim 9, wherein the poly(alkylene glycol) is a polyethylene glycol.

15. The method as claimed in claim 10, wherein the arylether(meth)acrylate of (poly)alkylene glycol is ethoxylated phenol tristyryl(meth)acrylate containing 25 moles of ethylene oxide.

16. The method as claimed in claim 1, wherein the weight ratio of the dispersant to the carbon nanotubes employed ranges from 0.6:1 to 1:1.

17. The method as claimed in claim 1, wherein the total mass of dispersant and carbon nanotubes accounts for 0.1 to 5% of the weight of the aqueous medium.

18. The method as claimed in claim 1, wherein the total mass of dispersant and carbon nanotubes accounts for 0.5 to 2% of the weight of the aqueous medium.

19. The method as claimed in claim 1, wherein the speed of the rotor is set to at least 1000 rpm.

20. The method as claimed in claim 1, wherein the width of the air gap between the rotor and the stator is lower than 1 mm.

21. The method as claimed in claim 1, wherein the rotor-stator system confers a shear of 1000 to 10⁹ s⁻¹.

22. A suspension capable of being obtained by the method as claimed in claim 1.

23. An article chosen from, polymer matrices, packing material for electronic components, inks for electrical connection between two electronic components, medical instruments, fuel hoses, adhesives, antistatic coatings, thermistors, or electrodes of light emitting diodes, photovoltaic cells or supercapacitances, made from a suspension as claimed in claim 22.
